(19) United States
(12) Patent Application Publication
(10) Pub. No.: US 2013/0137171 A1
(43) Pub. Date: May 30, 2013

(54) METHOD FOR PRODUCING ALGAE IN THIN

(71) Applicants: Lawrence V. Dressler, Cranston, RI (US); Alexander Chirkov, Lincoln, RI (US)

(72) Inventors: Lawrence V. Dressler, Cranston, RI (US); Alexander Chirkov, Lincoln, RI (US)

(21) Appl. No.: 13/525,272

(22) Filed: Jun. 15, 2012

Related U.S. Application Data
(63) Continuation-in-part of application No. 12/137,613, filed on Jun. 12, 2008.
(60) Provisional application No. 61/497,510, filed on Jun. 15, 2011.

(51) Int. Cl. C12M 1/00

(52) U.S. Cl. CPC C12M 21/02
USPC 435/292.1

(57) ABSTRACT

The present method transfers carbon dioxide in increased concentrations using perfluorodecalin for growth of algae in a photobioreactor. First, a perfluorodecalin solution is provided and mixed with a biological growth medium and a surfactant. The biological growth medium, perfluorodecalin solution, and surfactant mixture are then emulsified by circulation in a high-pressure emulsifier. The emulsified biological growth medium, perfluorodecalin solution, and surfactant mixture are then added to a photobioreactor containing algae capable of photosynthetically utilizing carbon dioxide. After adding carbon dioxide to the photobioreactor, the carbon dioxide dissolves in the perfluorodecalin solution at a higher concentration than in the growth medium. Conditions sufficient for the algae to perform photosynthesis using carbon dioxide from the perfluorodecalin solution are maintained thereby increasing the growth rate of the algae in increased concentration of carbon dioxide due to the increased solubility of carbon dioxide in the perfluorodecalin solution.

### CROSS REFERENCE TO RELATED APPLICATIONS

This application is related to and claims priority from earlier filed U.S. Provisional Application for Patent No. 61/497,510 filed Jun. 15, 2011, and earlier filed U.S. Non-Provisional patent application Ser. No. 12/137,613 filed Jun. 12, 2008, which are all incorporated herein by reference.

### BACKGROUND OF THE INVENTION

The invention generally relates to bioreactors. In particular the invention relates to a method of increasing the productivity and growth of algae in a thin film bioreactor More specifically, the invention is relates to methods and apparatus for large capacity and scalable bioreactors for use above-the-ground which includes thin film bioreactors. The thin film bioreactors increases the efficiency of algae production by reducing or minimizing the volume of water to a small amount required and a size of an area required for large scale algae production. The more efficient production of algae using a thin film bioreactor allows large scale algae production in much smaller areas with less water available than previously was feasible.

Algae are used in production of biofuel. Algae are low-cost/high-yield (30 times more energy per acre than land plants) feedstocks to produce biofuels. Algae have use as a renewable biomass source for the production of a diesel fuel substitute (biodiesel) and for electricity generation. Burning of fossil fuels in power plants is a primary contributor to excess carbon dioxide in the atmosphere, which has been linked to global climatic change. Release of carbon dioxide into the atmosphere can be significantly reduced by operation of algae fuel farms in tandem with fossil fuel plants to scrub CO2 from flue gases. If the algae are used to produce fuel, a mass culture facility reduces the CO2 emission from the power plant by approximately 99%.

Algae for use in production in biofuel are produced by photosynthesis. Photosynthesis may simply be defined as the conversion of light energy into chemical energy by living organisms. The raw materials are carbon dioxide and biological growth media; the energy source is sunlight; and the end-products are oxygen and (energy rich) carbohydrates, for example sucrose and starch. Algae are affected by its surroundings and the rate of photosynthesis is affected by the concentration of carbon dioxide, the intensity of light, and the temperature. A commonly used description of photosynthesis is: carbon dioxide+biological growth media+light energy→glucose+oxygen+biological growth media.

Photosynthesis occurs in two stages. In the first phase, light-dependent reactions or photosynthetic reactions (also called the Light reactions) capture the energy of light and use it to make high-energy molecules. During the second phase, the light-independent reactions (also called the Calvin-Benson Cycle) use the high-energy molecules to capture carbon dioxide and make the precursors of carbohydrates.

The rate of photosynthesis is affected by the concentration of carbon dioxide, the intensity of light, and the temperature. As carbon dioxide concentrations rise during photosynthesis, the rate at which sugars are made by the light-independent reactions increases until limited by other factors. RuBisCO, the enzyme that captures carbon dioxide in the light-independent reactions, has a binding affinity for both carbon dioxide and oxygen. When the concentration of carbon dioxide is high, RuBisCO will fix carbon dioxide. When the oxygen concentration is high, RuBisCO will bind oxygen instead of carbon dioxide through a process called photorespiration. Photorespiration lowers the efficiency of photosynthesis by removing carbon dioxide molecules from the Calvin-Benson Cycle. Therefore, in the presence of excess oxygen, the growth of algae will thus be inhibited.

Algae are organisms that can grow photosynthetically on carbon dioxide and sunlight, plus a minimum amount of nutrients. To grow algae in a bioreactor, such as a photobioreactor, the following factors may affect the process: media, such as fresh or salt biological growth media, and the physical conditions of the media, such as temperature and pressure; light as a source of energy for photosynthesis; and nutritional components, such as carbon dioxide, minerals, vitamins etc. The growth of algae is thus regulated by availability of light, nutritional components in the biological growth media, and physical condition of the system such as temperature and pressure. The process is also affected by by-products of photosynthesis, namely, oxygen, which is produced during the life cycle of algae. Accordingly, oxygen also limits the growth of algae in return.

In the prior art, carbon dioxide is introduced into a bioreactor to increase the growth rate of plant material, such as algae. In known photobioreactor systems, the algae obtain their carbon from carbon dioxide, often bubbled through the culture medium. The carbon dioxide is often introduced in the medium through sparging tubes or other suitable means positioned near the bottom of the photobioreactors. The bubbling of the carbon dioxide often serves a dual function in that it aids in the circulation of the algal culture.

U.S. Pat. No. 7,172,691 discloses that there are more than one means for introducing carbon dioxide into a reaction mixture. One method of adding carbon dioxide to the reaction mixture is by exposing the reaction mixture to air at its surface, and a portion of carbon dioxide from the air is dissolved in the reaction mixture in an open tank systems. Turning or mixing the reaction mixture increases exposure of the mixture to air and enhances dissolution of carbon dioxide into the reaction mixture. Other methods of introducing carbon dioxide into the reaction mixture can be employed including a carbon dioxide bubbler or jet into the reaction mixture at one or multiple inlets of the tank.

All nutritional factors, except carbon dioxide, could be delivered to algae in a bioreactor in any requested concentration without changes of any other physical characteristics of the system, such as temperature or pressure. However, carbon dioxide has a solubility limit in biological growth media. The increasing concentration of carbon dioxide in the bioreactor further limits physical solubility of carbon dioxide in biological growth media. This solubility can be increased by increasing partial pressure in the system. But, increasing the partial pressure has a negative effect on the growth rate of algae.

As indicated above, it is known that carbon dioxide has a limited solubility in biological growth media. When the reaction mixture is oversaturated with carbon dioxide, it exists in the biological growth media as a bubble, i.e. normal carbonation of biological growth media. However, the gas in bubble form is not available for consumption by algae. Accordingly, there are known limits to how much carbon dioxide can be delivered to growing algae.

One method of the prior art involves increasing the concentration of carbon dioxide in the air above the tank, such as by forming a sealed enclosure over the surface of the tank, and introducing carbon dioxide in the area over the surface and within the enclosure to form a carbon dioxide rich atmosphere above the surface of the reaction mixture. The drawback to this method is that increasing the concentration of carbon dioxide in the air does not necessarily increase directly the amount of carbon dioxide in the reaction mixture.

To overcome this limited solubility of carbon dioxide in biological growth media, emulsion perfluorocarbons (PFC) were introduced into the biological growth media. PFCs allow for increased gas solubility, are chemically inert, and have not been demonstrated to be biodegradable or been shown to be toxic to microorganisms. Gas solubility in PFCs follows Henry's law. Gas laden PFCs contacting microorganisms in the growth media increase the transfer of gases to the microorganisms and thereby increase the metabolic rates of the microorganisms. By emulsifying the PFCs, the surface area is increased allowing for increased contact with microorganisms and an even greater rate improvement. Examples of perfluorocarbons used for increasing the solubility of biological growth media are perfluorodecalin, perfluorohexane, perfluorobentane, and perfluorobenzene.

As an example, U.S. Pat. No. 5,637,499 provides a method to deliver industrial gases in increased concentrations to bacteria for growth in a bioreactor. The increase in solubility of industrial gases in a biological growth medium is accomplished by using vectors such as perfluorocarbon emulsions, as an additive to the medium. The increased concentration of industrial gases can increase the growth rates of bacteria in the growth medium.

Table 4 of the '499 patent, as shown in FIG. 1, demonstrates that gas solubility was increased in varying degrees with PFCs relative to controls. The '499 focuses on the productivity and growth of bacteria which is typically inhibited when using carbon dioxide. Therefore, the '499 patent does not disclose using perfluorodecalin for increasing the delivery of carbon dioxide to algae.

### BRIEF SUMMARY OF THE INVENTION

The present invention preserves the advantages of prior methods for increasing the productivity and growth of algae in photobioreactor. In addition, it provides new advantages not found in currently available methods for increasing the production and growth of algae in a photobioreactor and overcomes many disadvantages of such currently available methods. The present invention provides a method to deliver carbon dioxide in increased concentrations using perfluorodecalin for growth of algae in a photobioreactor to use in the production of biofuel. The method also increases fatty acids within algae when perfluorodecalin is used to increase the concentration of carbon dioxide. In addition, the present method uses perfluorodecalin to carry oxygen away from algae after photosynthesis.

The present invention transfers carbon dioxide in increased concentrations using perfluorodecalin for growth of algae in a photobioreactor. First, a perfluorodecalin solution is provided and mixed with a biological growth medium and a surfactant. The biological growth medium, perfluorodecalin solution, and surfactant mixture are then emulsified by circulation in a high-pressure emulsifier. The emulsified biological growth medium, perfluorodecalin solution, and surfactant mixture are then added to a photobioreactor containing algae capable of photosynthetically utilizing carbon dioxide. After adding carbon dioxide to the photobioreactor, the carbon dioxide dissolves in the perfluorodecalin solution at a higher concentration than in the growth medium. Conditions sufficient for the algae to perform photosynthesis using carbon dioxide from the perfluorodecalin solution are maintained thereby increasing the growth rate of the algae in increased concentration of carbon dioxide due to the increased solubility of carbon dioxide in the perfluorodecalin solution.

The present invention also consists of a photobioreactor system used in the method for increased production of algae. The photobioreactor system has a container for containing algae and a light within the container for photosynthesis. The photobioreactor system has a means for introducing emulsion containing biological growth medium, perfluorodecalin solution, and surfactant mixture to contact the algae. The photobioreactor system has a means for introducing carbon dioxide into the container such that the carbon dioxide dissolves in the perfluorodecalin solution at a higher concentration than in the growth medium and the carbon dioxide photosynthetically reacts with the algae in said container means in the presence of light. The photobioreactor has a means for controlling a temperature and agitation rate of the growth medium, perflourodecalin solution and algae within the photobioreactor to maintain conditions sufficient for the algae to perform photosynthesis using carbon dioxide from the perfluorodecalin solution, thereby increasing the growth rate of the algae in increased concentration of carbon dioxide due to the increased solubility of carbon dioxide in the perfluorodecalin solution. The photobioreactor has a means for circulating the emulsion within said container to facilitate photosynthesis of algae within said container.

It is therefore an object of the present invention to deliver carbon dioxide in increased concentrations using perfluorodecalin for growth of algae in a photobioreactor to use in the production of biofuel.

It is another object of the present invention to provide a method for increasing solubility of carbon dioxide in the biological growth media by using perfluorodecalin.

It is an object of the present invention to use perfluorodecalin to carry oxygen away from algae after photosynthesis to facilitate growth of the algae.

It is yet another object of the present invention to provide a method of increasing the fatty acid content of algae which is used in production of biofuels.

It is a further object of the present invention to provide a photobioreactor for use with the method for increasing the production of algae using perfluorodecalin.

It is another object of the present invention to provide a thin film bioreactor to increase the production of algae using less water and space.

### BRIEF DESCRIPTION OF THE DRAWINGS

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a prior art table from U.S. Pat. No. 5,637,499 disclosing the increase of solubility of carbon dioxide in a microbiological medium when using perfluorodecalin;

FIG. 2 is a prior art schematic view of a photobioreactor as an example of a photobioreactor for use in the method of the present invention;

FIG. 3 is a block diagram of the present invention;

FIG. 4 is a graph of algae growth in water or water with perfluorodecalin when carbon dioxide is added; and

FIG. 5 is a graph of change in partial pressure of carbon dioxide in water or water with the perfluorodecalin after algae is added.

FIG. 6 is perspective view of a single thin film bioreactor in another embodiment of the invention;

FIG. 7 is a perspective view of more than one thin film bioreactors of FIG. 6 stacked upon another to reduce space and water usage;

### DETAILED DESCRIPTION OF THE PREFERRED EMBODIMENTS

In accordance with the present invention, a new method for increasing the delivery of carbon dioxide in increased concentrations using perfluorodecalin for growth of algae in a photobioreactor. More specifically, the algae are produced within the photobioreactor for use in the production of biofuel.

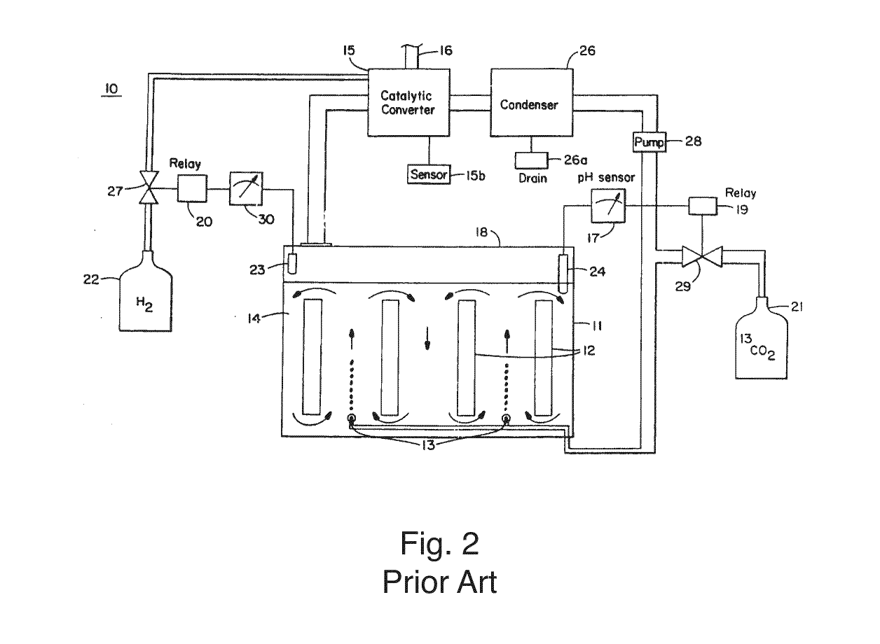

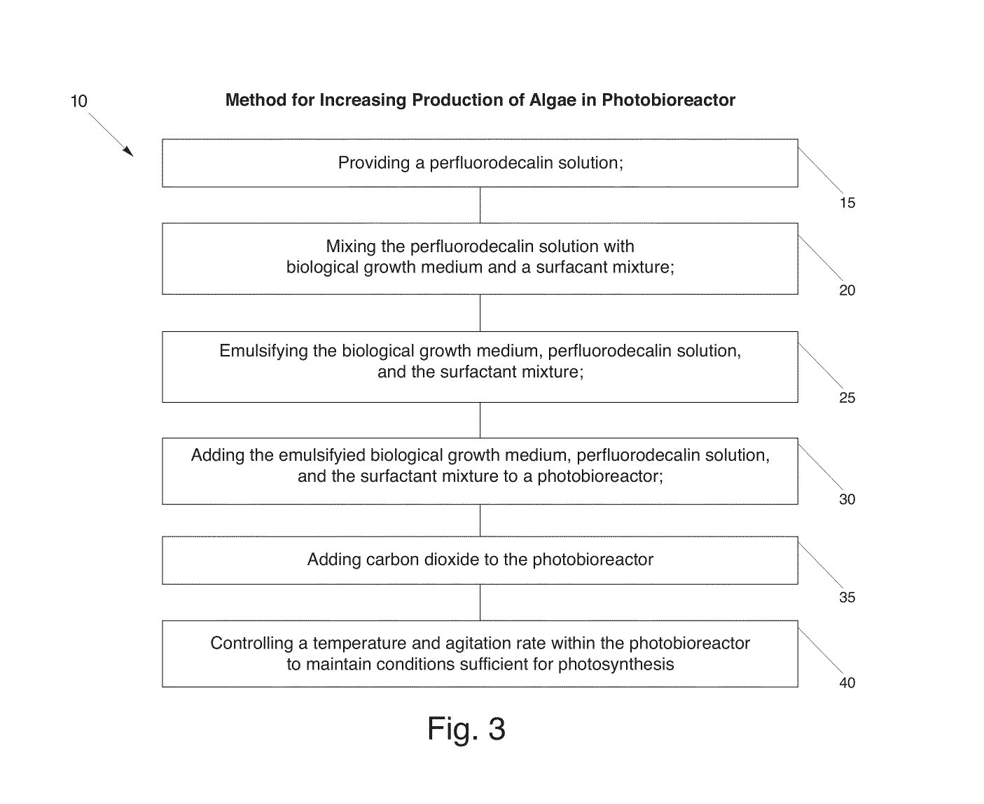

As shown in FIG. 3, the present invention is a method of transferring carbon dioxide in increased concentrations using perfluorodecalin for growth of algae in a photobioreactor 10. Algae is known for attracting and accumulating on its surface both carbon dioxide and oxygen as long as space is available. By way of example, a photobioreactor is used throughout this description but, by no means, is the photobioreactor the only bioreactor suited for production of algae for use in the present invention. By way of example only, a photobioreactor that is used in the prior art is illustrated at FIG. 2. By adding perfluorodecalin to the biological growth medium within the photobioreactor system, the solubility of carbon dioxide will be increased in the biological growth medium. The increased concentration of carbon dioxide is then available for use by the algae in photosynthesis and thus the productivity of algae will increase. Furthermore, since perfluorodecalin will also carry oxygen away from the algae, it is believed that perflourodecalin will further enhance the growth and fat content of the algae.

Perfluorodecalin can be used as 4% to up to 20% solution without significantly affecting the nutritional media for algae growth. Due to its small size, perfluorodecalin will be filtrated easily from algae during the harvesting. After simple recycling, it can be used again for algae growth. Perfluorodecalin is reusable and has an extended life.

Perfluorodecalin is capable of dissolving large amounts of oxygen and carbon dioxide in a biological growth medium and acts as the carrier of oxygen and carbon dioxide. Perfluorodecalin will tend to circulate in dependent areas and those areas where gas exchange is most diminished. Overall, the benefits of perfluorodecalin are improved gas exchange for use in the production of algae. To date, there is no known use of perfluorodecalin in a method of increasing the productivity and growth of algae in a photobioreactor system. The method of the present invention is further explained below.

Referring to FIG. 3, the present method begins by providing a perfluorodecalin solution 100 and mixing it with a biological growth medium and a surfactant 200. The biological growth medium is suited to support algae capable of photosynthetically utilizing carbon dioxide and the surfactant capable of being emulsified. It is contemplated other perfluorocarbons, other than perfluorodecalin, may be used in the current method. In a preferred embodiment, the biological growth medium is an aqueous solution, such as water.

The biological growth medium, perfluorodecalin solution, and surfactant mixture are then emulsified by circulation in a high-pressure emulsifier so that the perfluorodecalin solution is in the distributed state throughout the emulsified biological growth medium 300. In a preferred embodiment, the surfactant mixture contains phospholipids. The present method uses perfluorocarbons, preferably perfluorodecalin, or phospholipids or both chemicals to increase productivity and growth of algae.

The emulsified biological growth medium, perfluorodecalin solution, and surfactant mixture are then added to a photobioreactor containing algae capable of photosynthetically utilizing carbon dioxide 400. After adding carbon dioxide to the photobioreactor containing emulsified growth medium 500, perfluorodecalin solution, and surfactant mixture, the carbon dioxide dissolves in the perfluorodecalin solution at a higher concentration than in the growth medium.

Once photosynthesis begins in the photobioreactor, the temperature and agitation rate of the biological growth medium, perfluorocarbon solution and algae within the photobioreactor are maintained sufficiently for the algae to perform photosynthesis using carbon dioxide from the perfluorodecalin solution, thereby increasing the growth rate of the algae in increased concentration of carbon dioxide due to the increased solubility of carbon dioxide in the perfluorodecalin solution 600.

During photosynthesis in the photobioreactor, the perfluorodecalin releases carbon dioxide into the biological growth media for use by algae in photosynthesis. It is contemplated that the, in one embodiment, the perfluorodecalin is pretreated with carbon dioxide before entering the photobioreactor. After releasing the carbon dioxide, the perfluorodecalin absorbs oxygen produced as a byproduct of photosynthesis using perfluorodecalin to moves away from the algae. This release of carbon dioxide and absorption of oxygen by perfluorodecalin facilitates maintaining a steady state saturation level of carbon dioxide surrounding the algae.

In addition, by adding and regulating the perfluorodecalin for use in increasing the concentration of carbon dioxide, an increased production of fatty acids is provided in the algae. A higher fat content of algae is desirable in the production of alternative fuels, such as biodiesel. As a result, perfluorodecalin works as a carrier for transporting carbon dioxide to the algae and absorbing oxygen to move it away from algae. The method results in the increase of the growth rate and fat content of algae.

By maintaining a steady state saturation level of carbon dioxide, the growth rate and fatty acids of algae will increase. This algae with fatty acids is desirable for production of oils used in biofuels once removed from the photobioreator. To begin, the algae are harvested from the container by separating the algae from the emulsion containing perfluorodecalin solution. Once the algae are harvested, the perfluorodecalin solution is recycled from the container for future use. To assist in the production of biofuel, the oils are extracted from the algae for use in production of biofuel.

TABLE 1

| | Perfluorodecalin | |
|---|---|---|
| HOURS | Solution and Water | Water Only |
| 2 | 0.1 | 0.1 |
| 4 | 0.5 | 0.5 |
| 6 | 1.1 | 1.0 |
| 8 | 2.9 | 3.0 |
| 10 | 4.4 | 4.0 |
| 12 | 5.6 | 5.0 |
| 14 | 6.0 | 6.0 |
| 16 | 7.1 | 6.6 |
| 18 | 9.8 | 6.8 |
| 20 | 11.4 | 6.6 |
| 22 | 12.2 | 6.9 |
| 24 | 13.0 | 7.0 |
| 26 | 13.2 | 7.0 |
| 28 | 13.5 | 7.0 |
| 30 | 13.7 | 7.0 |
| 32 | 13.9 | 7.0 |
| 34 | 14.0 | 6.9 |
| 36 | 14.5 | 7.1 |
| 38 | 15.0 | 7.0 |
| 40 | 15.3 | 6.9 |
| 42 | 15.5 | 7.0 |
| 44 | 15.7 | 6.9 |
| 46 | 16.0 | 6.8 |
| 48 | 16.0 | 6.5 |

### Example 1

An experiment for testing the solubility of carbon dioxide in a biological growth media, such as water, with and without the perfluorodecalin solution was conducted with test results shown in Table 1. The experiment consisted of placing a sample of algae into two separate vessels. One test vessel contained water only and labeled “water only”. The second test vessel contained water and perfluorodecalin solution and labeled “perfluorodecalin solution and water”. Next, carbon dioxide was added to both vessels containing algae. Algae contained in each vessel were provided light and nutrients to grow in addition to the water or water and perfluorodecalin solution to simulate a bioreactor. Note, the entire time period for testing was 48 hours with testing being done every 2 hours.

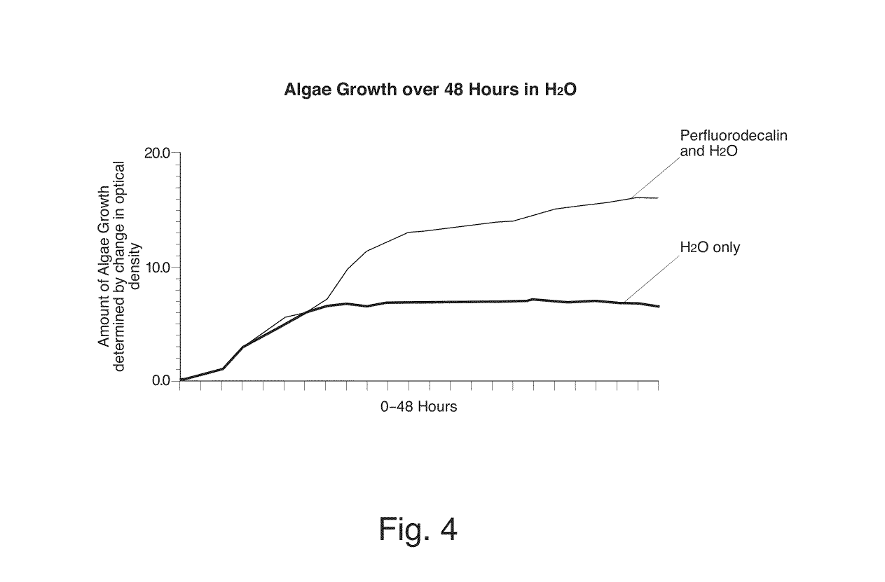

Referring to a graph in FIG. 4, the vessel with “water only” showed a continuous growth rate for the first 22 hours and then the growth rate for the algae stagnated. The vessel with “perfluorodecalin solution and water” maintained continuous growth of the algae throughout the 48 hours with a slight slow down around 46 hours. From reviewing the results of Table 1, the growth of algae in carbon dioxide inside the vessel containing “perfluorodecalin solution and water” over the 48 hour period was approximately 2.5 times better than the vessel containing “water only”.

A lab bench test was performed using an equipment to simulate a bioreactor produced the results in Table 1. Without being bound to any particular theory, it is believed that the growth rate of the algae would be greater than 2.5 times, possibly four times greater, using the perfluorodecalin solution and water inside a bioreactor setting.

TABLE 2

| | Perfluorodecalin | |
|---|---|---|
| HOURS | Solution and Water | Water Only |
| 2 | 98 | 98 |
| 4 | 98 | 98 |
| 6 | 98 | 92 |
| 8 | 98 | 90 |
| 10 | 98 | 83 |
| 12 | 98 | 78 |
| 14 | 98 | 70 |
| 16 | 98 | 67 |
| 18 | 98 | 64 |
| 20 | 98 | 60 |
| 22 | 98 | 56 |
| 24 | 97 | 50 |
| 26 | 97 | 48 |
| 28 | 97 | 46 |
| 30 | 97 | 43 |
| 32 | 96 | 40 |
| 34 | 95 | 36 |
| 36 | 96 | 32 |
| 38 | 96 | 30 |
| 40 | 95 | 29 |
| 42 | 96 | 29 |
| 44 | 96 | 29 |
| 46 | 96 | 28 |
| 48 | 96 | 28 |

### Example 2

An experiment for testing the amount of carbon dioxide that remains in a biological growth medium, such as water, with and without the perfluorodecalin solution after adding algae was conducted with test results shown in Table 2. One test vessel contained water only and labeled “water only”. The second test vessel contained water and perfluorodecalin solution and labeled “perfluorodecalin solution and water”. Next, carbon dioxide was added to both vessels.

After the carbon dioxide was added, a sample of algae was placed into the separate vessels. Algae contained in each vessel were provided light and nutrients to grow in addition to the water or water and perfluorodecalin solution to simulate a bioreactor. Note, the entire time period for testing was 48 hours with testing being done every 2 hours.

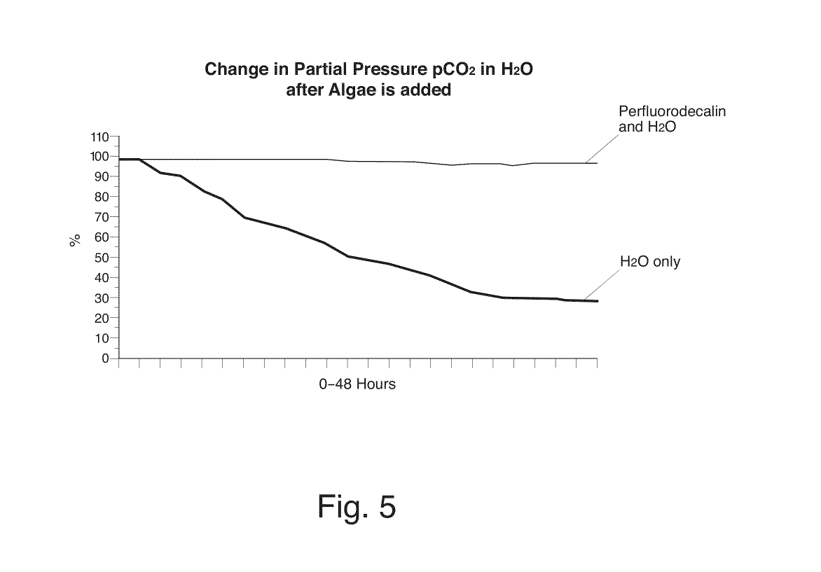

Referring to a graph in FIG. 5, the vessel with “water only” showed a continuous decline of partial pressure of carbon dioxide throughout the 48 hour period. The vessel with “perfluorodecalin solution and water” maintained a high partial pressure of carbon dioxide throughout the 48 hours. From reviewing the results of Table 2, the partial pressure of carbon dioxide inside the vessel containing “perfluorodecalin solution and water” over the 48 hour period was maintained, declining approximately 2%, while the vessel containing “water only” had a sharp decline beginning around 6 hours and dropping approximately 70%.

The present invention also consists of a photobioreactor system used in the method for increased production of algae. The photobioreactor system has a container for containing algae and a light within the container for photosynthesis. The photobioreactor system has a means for introducing emulsion containing biological growth medium, perfluorodecalin solution, and surfactant mixture to contact the algae. The photobioreactor system has a means for introducing carbon dioxide into the container such that the carbon dioxide dissolves in the perfluorodecalin solution at a higher concentration than in the growth medium and the carbon dioxide photosynthetically reacts with the algae in said container means in the presence of light. The photobioreactor has a means for controlling a temperature and agitation rate of the growth medium, perfluorodecalin solution and algae within the photobioreactor to maintain conditions sufficient for the algae to perform photosynthesis using carbon dioxide from the perfluorodecalin solution, thereby increasing the growth rate of the algae in increased concentration of carbon dioxide due to the increased solubility of carbon dioxide in the perfluorodecalin solution. The photobioreactor has a means for circulating the emulsion within said container to facilitate photosynthesis of algae within said container.

The photobioreactor is used for extracting algae for use in production of biofuels. The photobioreactor has a means for harvesting algae from said container and a means for recycling perfluorodecalin solution from said container for future use. Once the algae are retrieved, the photobioreactor may further include a means for extracting the oils from algae obtained from said container for use in production of biofuel.

In view of the foregoing, a new method for increasing the productivity and growth of algae in a biological growth photobioreactor system is disclosed. Perfluorodecalin is used in an emulsion to transfer increased concentration of carbon dioxide to increase the production and growth of algae in a photobioreactor system. To further enhance the growth of the algae, perfluorodecalin will also carry oxygen away from the algae. The method of increasing the productivity and growth of algae using perfluorodecalin overall reflects a significant improvement over prior art methods.

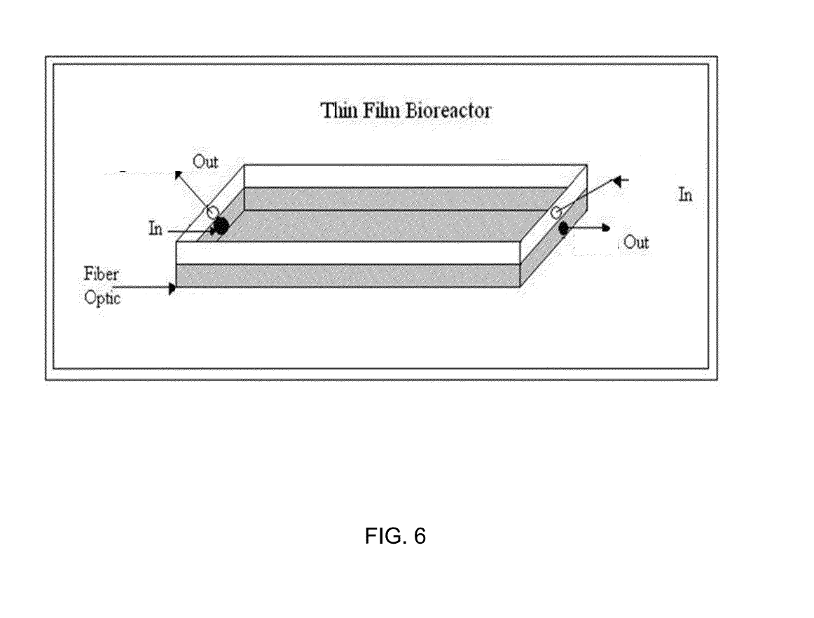

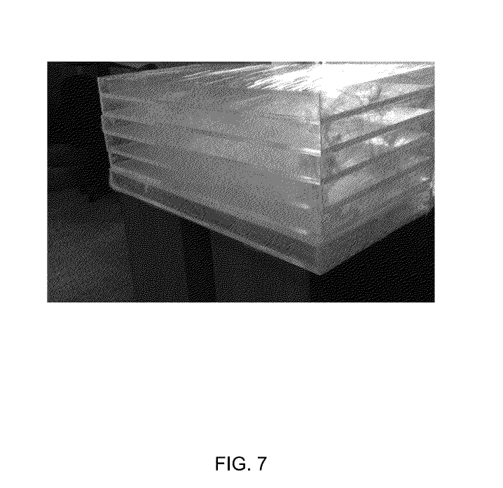

Referring to FIGS. 6-7, in another embodiment of the invention, the thin film bioreactors are disclosed which incorporate the advantages and benefits of the above-mentioned method for increasing the delivery of carbon dioxide in increased concentrations using perfluorcarbon, such as perfluorodecalin, for growth of algae in a photobioreactor. In particular the invention relates to a method of increasing the productivity and growth of algae in a thin film bioreactor More specifically, the invention is relates to methods and apparatus for large capacity and scalable bioreactors for use above-the-ground which includes thin film bioreactors. The thin film bioreactors increases the efficiency of algae production by reducing or minimizing the volume of water to a small amount required and a size of an area required for large scale algae production. The more efficient production of algae using a thin film bioreactor allows large scale algae production in much smaller areas with less water available than previously was feasible.

More specifically, the present invention relates to methods and apparatus for large capacity and scalable bioreactors for use above-the-ground which includes thin film bioreactors containing algae culture, water (also known as biological growth medium) and nutrient supplements to assist in photosynthesis of algae. The liquid depth on or in each unit is predetermined based on, but not limited to, temperature, algal species, nutrient level, and light duration and intensity. The ability of this thin film bioreactor to provide large scale algal production on minimal water usage will provide an innovative method of using algae to replace fossil fuels as a bioenergy source on a competitive basis.

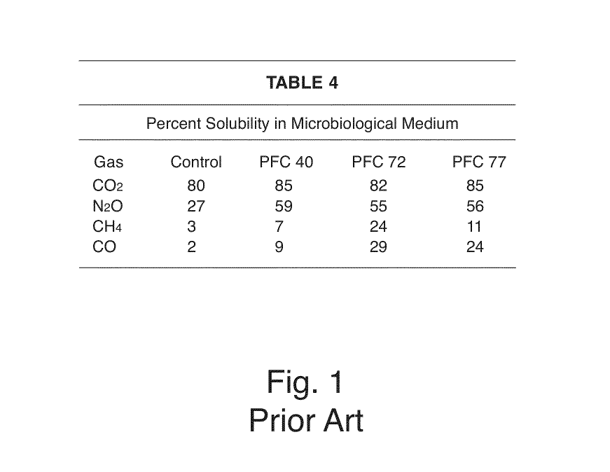

The invention is a thin film bioreactor which provides an ability to grow algae which allows for the low cost commercial scale up of algal production with the minimal amount of water usage. As illustrated in FIG. 1 below, a thin film bioreactor for the growth of algal biomass includes a container for containing the required water medium and a absorber solution or perfluorocarbon solution. More specifically, the container may be a tray generally defining an overall low profile square or rectangular shape.

The thin film bioreactor is comprised of a flat shallow container or tray with raised edges used for holding liquid and other materials. The liquid depth in each tray is predetermined based on, but not limited to, temperature, algal species, nutrient level, and light duration and intensity. Each tray shall include, but not be limited to, an artificial, such as LEDs, or natural light, such as sunlight, supply distributed from above or below the biological growth medium. Each tray may be operated as a plug flow reactor. Each individual tray can serially communicate with another so as to achieve a “plug flow” reactor of exceeding volumetric capacity.

The liquid containing water, nutrients and algae is pumped to the inlet of the thin film bioreactor. The perflurocarbon gas exchange medium enters the unit in a concurrent or countercurrent flow. As the algae, water and nutrients flow through or mix within the tray, the algae growth increases exponentially. Based upon differences in partial pressure of perfluorocarbon solution (also known as absorber solution), it will release carbon dioxide and absorb oxygen. This growth consumes CO2 (carbon dioxide) and produces O2 (oxygen). The CO2 (carbon dioxide) is provided via the absorber solution and the O2 (oxygen) is absorbed by the absorber solution. The algae will continue growing and achieve rates facilitated by the energy supplied by the lighting mechanism chosen.

The contents of the bioreactor exiting the unit will overflow into a harvesting device to remove the algal biomass. The liquid contents exiting the harvesting device is recycled to the thin film bioreactor. The absorber solution is collected and re-exposed to a CO2 using the process outlined in U.S. patent application Ser. No. 12/137,613 filed Jun. 12, 2008 entitled METHOD FOR PRODUCING ALGAE IN PHOTOBIOREACTOR. The absorber solution is collected base on differences in density with water, insolubility in water and saturated with carbon dioxide for next round of production. Based upon information and belief, the use of the absorber solution, allows the water needs of this process and thin film bioreactor to be reduced by as much as 90%. Based upon information and belief, the thin-film bioreactor using perfluorodecalin will produce more algae per square inch in volume of water achieving about 50 grams per liter of water as opposed to 2-10 grams per liter of water.

FIG. 6 is a side view of the thin-film bioreactor system having a tray. The process shows the algae & water going into the reactor on the right. A thin film of the algae, water and nutrients is formed over the perfluorcarbon solution. In this situation, the algae, using photosynthesis, is expected to grow within the biological growth medium or emulsion including the water and nutrients. In one embodiment, the tray is 10 cm in height with biological growth medium, water having algae and nutrients occupying approximately 6-7 cm in height within an upper portion of the tray, above the peflurodecalin solution, and the perfluorodecalin solution occupying approximately 0.1 cm in height within a lower portion of the tray. It should be noted that the perfluorodecalin solution is substantially transparent to permit light to pass therethrough more easily than biological growth medium. Also, the absorber solution is twice as dense as water which makes it rest within the lower portion of the try. The algae in water exits on the left where the algae is separated from the water. The water is then reused in the bioreactor. The perfluorcarbon solution presaturated with carbon dioxide enters on the left for the purpose of feeding the algae in the thin film. The perfluorcarbon solution exits on the right. A fiber optic cable is attached to tray which provides artificial light for the bioreactor. Alternatively, it should be noted that the algae, water, nutrients, and perflurodecalin solution may enter and exit through a primary aperture having a closure means therein for regulating the addition or removal of the algae, biological growth medium, nutrient supplements, and perfluorodecalin solution.

Referring to FIG. 7, the bioreactor may be configured in a stack and thus the fiber optic on the bottom of one reactor or tray can also provide light to the top of the reactor it is stacked above. In one embodiment, each tray may hold 100 liters of water. For example, ten stacked trays may hold 1000 liters of water or one cubic meter of water in combination. It should be noted that the trays may also include covers having a light reflective material, such as mirrors. In case of stacking trays on one another, the bottom wall of the first tray and a last tray cover must have light reflecting laying to maintain photon trap.

The oxygen with residual carbon dioxide exits the top of the tray. The oxygen produced and during algae growth could be collected and utilized. This gas stream can be utilized as direct feed into an auxiliary, existing or supplemental combustor as an oxygen supply, thereby, replacing or reducing the nitrogen contained in the air supplied to the combustion chamber. The thin film bioreactor can be connected in series with expanding or contracting segments or in parallel or any combination therein as required by the reaction or growth kinetics or models. The thin film bioreactor is “closed” allowing for the atmosphere of each reactor to be controlled and monitored.

It would be appreciated by those skilled in the art that various changes and modifications can be made to the illustrated embodiments without departing from the spirit of the present invention. All such modifications and changes are intended to be within the scope of the present invention.

What is claimed is:

1. A thin-film bioreactor system for increasing production of algae, comprising:
container means for containing algae;
means for introducing light into said container means to facilitate photosynthesis;
means for introducing emulsion containing biological growth medium having algae and nutrient supplements to assist in the growth of algae;
means for introducing perfluorocarbon solution pre-saturated with carbon dioxide at a high concentration level;
light reflecting material lining one or more surfaces of the container means to facilitate photosynthesis to grow algae within the biological growth medium; and
whereby increasing the growth rate of the algae in increased concentration of carbon dioxide due to the increased solubility of carbon dioxide in the perfluorocarbon solution.

2. The thin-film bioreactor system of claim 1, wherein the perflurocarbon solution is perfluordecalin solution.

3. The thin-film bioreactor system of claim 1, wherein the light is natural light source or an artificial light source.

4. The thin-film bioreactor system of claim 1, wherein the container means is a tray with having at least a bottom wall and four side walls.

5. The thin-film bioreactor system of claim 1, wherein the tray has an upper portion and a lower portion.

6. The thin-film bioreactor system of claim 1, wherein the tray defines a low profile.

7. The thin-film bioreactor system of claim 1, wherein the tray has approximate dimensions of ten centimeters in height, one meter in width, and one meter in length.

8. The thin-film bioreactor system of claim 7, wherein the dimensions of the tray has a ratio of 0.1 height to 1 width to 1 length.

9. The thin-film bioreactor system of claim 1, wherein one or more trays may be stacked upon one another to reduce usage of space.

10. The thin-film bioreactor system of claim 3, wherein the means for introducing light from a light source having 400-500 nm wavelength into the container means is a fiber optic cable dispersed within a material of a bottom wall of the tray.

11. The thin-film bioreactor system of claim 10, wherein the material of the bottom wall of the tray is glass material.

12. The thin-film bioreactor system of claim 1, wherein the tray defines a primary aperture having a closure means therein for regulating the addition or removal of the algae, biological growth medium, nutrient supplements, and perfluorodecalin solution.

13. The thin-film bioreactor system of claim 3, wherein the means for introducing light from a light source is positioned above the container means.

14. The thin-film bioreactor system of claim 4, wherein the tray has a means for introducing light embedded within the bottom wall and light reflecting material on one or more side walls to create a photon trap.
